(19) United States
(12) Patent Application Publication
(10) Pub. No.: US 2023/0263851 A1
(43) Pub. Date: Aug. 24, 2023

(54) PROTECTIVE FORMULATIONS

(71) Applicant: Eric Matthew STROUD, Oak Ridge, NJ (US)

(72) Inventor: Eric Matthew STROUD, Oak Ridge, NJ (US)

(21) Appl. No.: 18/172,618

(22) Filed: Feb. 22, 2023

Related U.S. Application Data
(60) Provisional application No. 63/312,776, filed on Feb. 22, 2022.

(51) Int. Cl. A61K 36/534; A61K 33/18; A61K 31/045; A61K 9/107; A61K 9/00; A61K 8/9783; A61K 8/20; A61K 8/34; A61Q 17/00; A61Q 17/04

(52) U.S. Cl. CPC A61K 36/534; A61K 33/18; A61K 31/045; A61K 9/1075; A61K 9/0014; A61K 8/9783; A61K 8/20; A61K 8/34; A61Q 17/005; A61Q 17/04

(57) ABSTRACT

The present disclosure generally relates to novel cosmetic and/or pharmaceutical formulations and methods of using them. The present formulations contain protective compounds that have been found to significantly reduce the dermal irritation following contact with stinging Cnidaria. These formulations are applied to the skin prior to entering the environment of Cnidaria. In some embodiments, the protective compounds include a rubidium salt, a monocyclic monoterpenoid, and C₇ to C₉ aliphatic alcohols.

### CROSS REFERENCE TO RELATED APPLICATIONS

This application claims priority benefit of U.S. Provisional Application No. 63/312,776 filed on Feb. 22, 2022, the contents of which are incorporated by reference herein in their entirety.

### FIELD OF THE DISCLOSURE

The present disclosure generally relates to novel cosmetic and/or pharmaceutical formulations and methods of using them. The present formulations contain protective compounds that have been found to significantly reduce the dermal irritation following contact with stinging Cnidaria. These formulations are applied to the skin prior to entering the environment of Cnidaria. In some embodiments, the protective compounds include one or more of a rubidium salt, a monocyclic monoterpenoid, and C₇ to C₉ aliphatic alcohols.

### BACKGROUND

Cnidarians are invertebrates of the phylum Cnidaria and include the corals, sea anemones, hydrae, and jellyfish organisms that possess stinging cells. Nematocysts, also called cnidae (the plural), are specialized the specialized stinging cells in these organisms that are used to capture prey and as a defense against predators. A nematocyst or enidocyte cell fires a structure that contains a toxin within the cnidocyst; this is responsible for the stings delivered by a cnidarian. These microscopic structures attach to human skin after contact and may envenomate the skin instantly, after rubbing or scratching, or after application of certain chemical compounds. Thousands of microscopic nematocysts may fire from physical contact with a single Cnidarian. Depending on the species and the quantity of envenomation, some stings are extremely painful and may even result in shock or death. There is a need for topical skin formulations to reduce or eliminate the effects of the nematocyst toxins in humans. The present disclosure is directed to these, as well other, important ends.

### SUMMARY OF THE DISCLOSURE

Provided herein are novel cosmetic and/or pharmaceutical formulations that contain protective compounds that have been found to significantly reduce the dermal irritation following contact with stinging Cnidaria.

In a first aspect, the present disclosure provides a topical formulation for protecting a mammal against Cnidaria toxins comprising at least about 0.01% w/w of an active component, the active component comprising one or more protective active compounds independently selected from monocyclic monoterpenoids; rubidium salts; and C₇-C₉ aliphatic alcohols; and a carrier component for maintaining the active component on the skin of the mammal.

In a second aspect, the present disclosure provides a method for protecting a mammal against Cnidaria toxins comprising applying a topical formulation as described herein to the skin of the mammal.

### BRIEF DESCRIPTION OF THE FIGURES

FIG. 1 shows the results of the in vivo study described in Example 2 herein.

### DETAILED DESCRIPTION OF THE DISCLOSURE

In a first aspect, the present disclosure provides a topical formulation [“Formulation 1”] for protecting a mammal against Cnidaria toxins comprising:
at least about 0.01% w/w of an active component, the active component comprising one or more protective active compounds independently selected from monocyclic monoterpenoids; rubidium salts; and C₇-C₉ aliphatic alcohols; and
a carrier component for maintaining the active component on the skin of the mammal.

The present disclosure further provides the following embodiments of Formulation 1:
1.1 Formulation 1, wherein the active component comprises one or more compounds selected from:
   a monocyclic monoterpenoid selected from DL menthol, α-terpineol, limonene, thymol, menthol, carvone, eucalyptol, perillaldehyde, p-menthane, an α, β, γ, δ-terpinene, α-phellandrene, β-phellandrene and hinokitiol in a concentration of from about 0.01% w/w to about 5.0% w/w, e.g. from about 0.1% w/w to about 5.0% w/w, e.g. from about 0.01% w/w to about 3.0% w/w, e.g. from about 0.25% w/w to about 1.5% w/w, e.g. about 0.25% w/w, about 0.50% w/w, about 0.75% w/w, about 1.0% w/w, about 1.25% w/w or about 1.50% w/w;
   a rubidium halide salt in a concentration of from about 0.01% w/w to about 3.0% w/w, e.g. from about 0.10% w/w to about 3.0% w/w, e.g. from about 0.50% w/w to about 1.5% w/w, e.g. about 0.50% w/w, about 0.75% w/w, about 1.0% w/w, 1.25% w/w or about 1.50% w/w; and
   a C₇-C₉ aliphatic alcohol selected from n-heptanol, n-octanol and n-nonanol in a concentration of from about 0.01% w/w to about 3.0% w/w, e.g. from about 0.10% w/w to about 3.0% w/w, e.g. from about 0.25% w/w to about 1.5% w/w, e.g. about 0.25% w/w, about 0.50% w/w, about 0.75% w/w, about 1.0% w/w, about 1.25% w/w or about 1.50% w/w.
1.2 Formulation 1.1, wherein the active component comprises one or more compounds selected from: DL menthol; rubidium iodide; and a C₇-C₉ aliphatic alcohol selected from n-heptanol, n-octanol and n-nonanol.
1.3 Any of Formulations 1 and 1.1-1.2, wherein the active component comprises rubidium iodide.
1.4 Any of Formulations 1 and 1.1-1.2, wherein the active component comprises DL menthol.
1.5 Any of Formulations 1 and 1.1-1.2, wherein the active component comprises one or more of n-heptanol, n-octanol and n-nonanol.
1.6 Any preceding Formulation, wherein the active component comprises from about 0.01% w/w to about 90% w/w of the formulation.
1.7 Formulation 1 or 1.1, wherein the active component comprises a combination of a monocyclic monoterpenoid selected from DL menthol, α-terpineol, limonene, thymol, menthol, carvone, eucalyptol, perillaldehyde, p-menthane, an α, β, γ, δ-terpinene, α-phellandrene, β-phellandrene, and hinokitiol, and a rubidium halide salt selected from rubidium iodide, rubidium chloride, rubidium bromide, and rubidium fluoride.
1.8 Formulation 1 or 1.1, wherein the active component comprises a combination of a monocyclic monoterpenoid selected from DL menthol, α-terpineol, limonene, thymol, menthol, carvone, eucalyptol, perillaldehyde, p-menthane, an α, β, γ, δ-terpinene, α-phellandrene, β-phellandrene and hinokitiol, and a C₁-C₉ aliphatic alcohol elected from n-heptanol, n-octanol and n-nonanol.
1.9 Formulation 1 or 1.1, wherein the active component comprises a combination of a rubidium halide salt and a C₇-C₉ aliphatic alcohol n-heptanol, n-octanol and n-nonanol.
1.10 Formulation 1.7, wherein the monocyclic monoterpenoid comprises DL menthol, and the rubidium halide salt comprises rubidium iodide.
1.11 Formulation 1.8, wherein the monocyclic monoterpenoid comprises DL menthol, and the C₇-C₉ aliphatic alcohol comprises one or more of n-heptanol, n-octanol and n-nonanol.
1.12 Formulation 1.9, wherein the rubidium halide salt comprises rubidium iodide, and the C₇-C₉ aliphatic alcohol comprises one or more of n-heptanol, n-octanol and n-nonanol.
1.13 Any of the preceding Formulations, wherein the carrier component comprises an aqueous (water) phase and a nonaqueous (oil) phase.
1.14 Formulation 1.13, wherein the carrier component comprises an oil-in-water emulsion.
1.15 Any of the preceding Formulations 1 and 1.1-1.12, wherein the carrier component comprises or consists of a nonaqueous (oil) phase.
1.16 Any of the preceding Formulations, wherein the carrier component is selected from a skin moisturizer formulation; an oil-in-water emulsion optionally comprising one or more sunscreen compounds; a dimethicone-based gelee (wax) formulation; an after-sun lotion, a “chemical free” or mineral sunscreen, a sprayable anhydrous (oil and alcohol) formulation, a sprayable milk, and a balm.
1.17 Any of the preceding Formulations 1 and 1.1, wherein the formulation comprises an aqueous (water) phase and a non-aqueous phase; the carrier component is a skin moisturizer formulation; and the active component comprises rubidium iodide in an amount of about 0.5% w/w to about 1.0% w/w, in the water phase.
1.18 Any of the preceding Formulations 1 and 1.1, wherein the carrier component is an oil-in-water emulsion; the active component comprises rubidium iodide in an amount of from about 0.5% w/w to about 1.5% w/w; e.g. about 1.0% w/w, in the water phase; and the carrier component optionally contains one or more sunscreen compounds
1.19 Any of the preceding Formulations 1 and 1.1, wherein the carrier component is a dimethicone-based gelee (wax) formulation; and the active component comprises DL menthol and a C₇-C₉ aliphatic alcohol selected from one or more of n-heptanol, n-octanol and n-nonanol, each incorporated into the carrier component; for example at a concentration of about 0.01% w/w to about 3.0% w/w.
1.20 Any of the preceding Formulations 1 and 1.1, wherein the carrier component is an after-sun lotion comprising an aqueous (water) phase and a non-aqueous (i.e., oil) phase; and the active component comprises rubidium iodide in an amount of from about 0.5% w/w to about 1.5% w/w; e.g. about 1.0% w/w in the water phase, and DL menthol in an amount of from about 0.2% w/w to about 1.0% w/w; e.g. about 0.5% w/w in the oil phase.

The topical formulations described herein can be several forms, including a topical lotion, gel, cream, wax, sunscreen, spray, balm, oil, moisturizer, ointment, after sun product, or similar cosmetic formulation. The present formulations are intended for application to any areas of the human body that may be exposed to stinging Cnidarians, including the face, arms, hands, torso, legs, and feet.

Protective Active Agents

The formulations described herein include one or more protective active agents (or protective active compounds). As used herein, the term “protective active agent” (or “protective active compound”) is intended to mean a compound that protects against dermal irritation following contact with stinging Cnidaria; i.e., a compound that significantly reduces the dermal irritation following contact with stinging Cnidaria when applied to the skin prior to contact with Cnidaria. In some embodiments, the protective active agent(s) are selected from a rubidium salt; a monocyclic monoterpenoid; and an aliphatic alcohol of having from 7 to 9 carbon atoms: i.e., a C₇-C₉ aliphatic alcohol.

In some preferred embodiments, the rubidium salt is a rubidium halide, for example rubidium iodide, rubidium chloride, rubidium bromide, or rubidium fluoride. In some preferred embodiments, the protective active agent comprises or consists of rubidium iodide, alone or in combination with another protective active agent.

In some preferred embodiments, the monocyclic monoterpenoid is selected from DL menthol (i.e., racemic menthol), α-terpineol, limonene, thymol, menthol, carvone, eucalyptol, and perillaldehyde. Selection of monocyclic monoterpenoid can be made on the basis of various factors, including efficacy, formulation capability, odor, and inherent dermal irritation. In some preferred embodiments, the monocyclic monoterpenoid comprises or consists of DL-menthol, alone or in combination with another protective active agent.

In some embodiments, the C₇-C₉ aliphatic alcohol is selected from n-heptanol, n-octanol and n-nonanol. In some preferred embodiments, the C₇-C₉ aliphatic alcohol comprises or consists of n-heptanol, alone or in combination with another protective active agent.

In some embodiments, the formulations of the present disclosure include more than one protective active agent.

The protective active agent(s) can be present in the formulation an amount of from about 0.1% w/w to about 90% w/w of the formulation. In some embodiments, the protective active agent(s) can be present in the formulation an amount of from about 0.01% w/w to about 5.0% w/w, e.g. about 0.1% w/w to about 5.0% w/w, e.g. about 0.01% w/w to about 3.0% w/w, e.g. from about 0.10% w/w to about 3.0% w/w, e.g. from about 0.25% w/w to about 1.5% w/w, about 0.25% w/w, about 0.50% w/w, e.g. about 0.50% w/w to about 1.5% w/w, e.g. about 0.25% w/w, about 0.50% w/w, about 0.75% w/w, about 1.0% w/w, about 1.25% w/w, about 1.50% w/w, about 1.75% w/w, about 2.0% w/w, about 2.25% w/w, abut 2.5% w/w, about 2.75% w/w, or about 3.0% w/w, about 5%% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 70% w/w, about 75% w/w, about 80% w/w, about 85% w/w or about 90% w/w of the formulation.

Additional Actives

In some embodiments, the present formulations include one or more optional active compounds. As used herein, the term “optional active compound” is intended to mean a compound that confers a benefit other than reducing the dermal irritation following contact with stinging Cnidaria when applied to the skin. Examples of optional active compounds include sunscreen agents, moisturizers, hydrating agents, and the like.

In some embodiments, the present formulations comprise after sun lotions that include one or more protective active agents for reducing the dermal irritation following contact with stinging Cnidaria as described herein. After sun lotions are used to moisturize and/or rehydrate skin following exposure to the sun. Numerous after sun lotions are commercially available, and their compositions are readily available both on the products and in the literature. Typical ingredients of after sun lotions in addition to moisturizing and hydrating compounds, include solvents, preservatives, thickening agents and emulsifiers such as glycerin, cetyl palmitate, ethylhexyl palmitate, ethylhexyl glycerin, myristyl alcohol, polysorbate 20, modified polyethylene glycols such as PEG-100 stearate, glyceryl stearate SE, mineral oil, palmitic acid, stearic acid, coconut oil, cetyl alcohol, sorbitol, Cocoa Seed Butter, tocopherol acetate, aloe vera, aloe barbadensis leaf juice, polymers such as Acrylates/C10-30 alkyl acrylate crosspolymer, polymethylsilsesquioxane, PVM/MA copolymer, etc.

In some preferred embodiments, the topical formulation for protecting a mammal against Cnidaria toxins is an after sun composition as described above, comprising one or more protective active compounds as described herein, e.g. rubidium iodide (RuI) in a concentration of about 0.1% w/w to about 3% w/w; e.g., about 0.50% w/w to about 1.5% w/w; e.g. about 1.0% w/w in the water phase of the formulation, and DL menthol in the oil phase of the formulation in a concentration of about 0.1% w/w to about 5% w/w; e.g., about 0.5% w/w.

In some embodiments, the present formulations comprise skin moisturizing formulations that include one or more protective active agents for reducing the dermal irritation following contact with stinging Cnidaria as described herein. Skin moisturizers are typically oil-in-water emulsions that contain moisturizing compounds such as occlusives, which hold water in after it has been supplied either by the moisturizer or by soaking in water; humectants that function to attract water, drawing it up from the dermis and, to a limited extent, in humid conditions, from the air; and emollients, which fill in rough spots and make skin feel smooth but don't affect the water content. Numerous occlusives are known in the art, and include, for example, petrolatum and the other oily substances such as cetyl alcohol (and other fatty alcohols), lanolin, lecithin, mineral oil, paraffin, stearic acid, and dimethicone and cyclomethicone, which are silicones that function as occlusives. Numerous humectants are known in the art, and include, for example, glycerin, honey, propylene glycol, panthenol (or vitamin B₅), sorbitol, proteins and urea. Numerous emollients are known in the art, and include, for example, dimethicone and alcohols such as octyldodecanol.

In some preferred embodiments, the topical formulation for protecting a mammal against Cnidaria toxins is a skin moisturizer composition as described above, comprising one or more protective active compounds as described herein, e.g. rubidium iodide (RuI) in a concentration of about 0.1% w/w to about 3% w/w; e.g., about 0.50% w/w to about 1.0% w/w; e.g. about 0.75% w/w in the water phase of the formulation.

In some embodiments, the present formulations comprise sunscreen formulations that include one or more protective active agents for reducing the dermal irritation following contact with stinging Cnidaria as described herein.

Sunscreen composition are typically oil-in-water emulsions that contain one or more UV-absorbing or blocking (sunscreen) compounds. Typical sunscreen formulations contain:
about 20% w/w active ingredients—i.e., compounds that block or absorb UV light;
about 55% formulation stabilizers—i.e. solvents, preservatives, thickening agents and emulsifiers;
about 23% sensory enhancers, i.e., compounds that improve the feel or scent of the formula, such as fragrances, moisturizers and emollients; and
about 2% added extras—i.e., compounds that do not influence how the sunscreen works, such as aloe and blue-light protectors.

Myriad sunscreen formulations and their compositions, including sunscreen compounds, are known in the art. See, e.g. Boerner, K., “What's in sunscreen, and how does it protect your skin from the sun's rays?” Chem. & Eng. News, available at https://cen.acs.org/business/consumer-products/What-in-sunscreen-and-how-does-it-protect-your-skin-from-the-sun-rays/99/i27, incorporated by reference herein.

Sunscreen formulations are rated by their sun protection factor (SPF), which is a measure of how much solar energy (UV radiation) is required to produce sunburn on protected skin (i.e., in the presence of sunscreen formulation) relative to the amount of solar energy required to produce sunburn on unprotected skin. As the SPF value increases, sunburn protection increases. In some preferred embodiments, the present formulations comprising sunscreen formulations have a SPF of at least 15.

In some preferred embodiments, the topical formulation for protecting a mammal against Cnidaria toxins is an oil-in-water emulsion sunscreen as described above, comprising one or more protective active compounds as described herein, e.g. comprising rubidium iodide (RuI) in a concentration of about 0.1% w/w to about 3% w/w; e.g., about 0.50% w/w to about 1.5% w/w; e.g. about 1.0% w/w, incorporated into the water phase after emulsification.

In some preferred embodiments, the topical formulation for protecting a mammal against Cnidaria toxins is a dimethicone-based gelee (wax) with n-heptanol and dl-menthol (racemic) incorporated directly into the oil phase, for example in a concentration of about 0.01% w/w to about 3.0% w/w.

Carriers

Suitable pharmaceutically acceptable components for the carrier component of the present compositions include, in addition to the components of after sun lotions, sunscreen formulations and skin moisturizing formulations discussed above, include one or more of fatty acid esters, fatty acids and salts thereof, fatty alcohols (such as lauryl, myristyl stearyl, cetyl or cetostearyl alcohol), fatty amines, fatty amides, glycerides (e.g., mono-, di- and tri-glycerides), hydrogenated fats, glycolipids, steroids, natural and synthetic waxes, polyethylene glycol (PEG) or derivatives, and the like. Specific examples include, but are not limited to, hydrogenated vegetable oil, hydrogenated cottonseed oil, hydrogenated castor oil, stearic acid, cocoa butter, glyceryl behenate, glyceryl dipalmitostearate, and stearyl alcohol. Mixtures of mono-, di- and tri-glycerides and mono- and di-fatty acid esters of polyethylene glycol are also suitable fatty materials. Suitable waxes and wax-like materials include natural or synthetic waxes, hydrocarbons, and normal waxes. Specific examples of waxes include beeswax, glycowax, castor wax, carnauba wax, paraffins and candelilla wax.

Polyethylene glycol or its derivatives may include PEG 200, PEG 300, PEG 400, PEG 600, PEG 1000, PEG 4000, PEG 6000, PEG 8000, PEG 12000, PEG 20000, polyglycolyzed glycerides, polyethylene glycol-polyoxyethylenes, polyethylene glycol polypropylenes, and polyethylene glycol-polyoxypropylenes.

Polymer components can include poloxamers, PVA/MA copolymers, Acrylates/C10-30 alkyl acrylate crosspolymer, polymethylsilsesquioxane, and other polymers known to be useful in topical formulations.

The pharmaceutical compositions provided herein may be provided any form suitable for topical administration including topical lotions, gels, creams, waxes, sunscreens, sprays, balms, oils, moisturizers, ointments, after sun products, sunscreen products, skin moisturizers or similar cosmetic formulations. Suitable ointment vehicles include oleaginous or hydrocarbon bases, including such as lard, benzoinated lard, olive oil, cottonseed oil, and other oils, white petrolatum; emulsifiable or absorption bases, such as hydrophilic petrolatum, hydroxystearin sulfate, and anhydrous lanolin; water-removable bases, such as hydrophilic ointment; water-soluble ointment bases, including polyethylene glycols of varying molecular weight; emulsion bases, either water-in-oil (W/O) emulsions or oil-in-water (OAV) emulsions, including cetyl alcohol, glyceryl monostearate, lanolin, and stearic acid (see, Remington's Pharmaceutical Sciences, Mack Publishing Company, Easton, Pa., 15th Edition, 1975 or later editions thereof, incorporated herein by reference for all purposes). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Further pharmaceutically acceptable carrier components and excipients suitable for use in the topical formulations provided herein can include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, penetration enhancers, cryoprotectants, lyoprotectants, thickening agents, inert gases, and sequestering or chelating agents.

In a second aspect, the present disclosure provides a Method [Method 1] for protecting a mammal against Cnidaria toxins comprising applying to the skin of the mammal a composition according to any of the Formulations described herein, for example Formulations 1 and 1.1-1.20.

In a third aspect, the present disclosure provides a Method [Method 2] for preparing a topical formulation for protecting a mammal against Cnidaria toxins comprising: providing a carrier component or carrier base as described herein; and combing the carrier component or carrier base with at least about 0.01% w/w of an active component, the active component comprising one or more compounds independently selected from monocyclic monoterpenoids; rubidium salts; and C₇-C₉ aliphatic alcohols. In some embodiments, the carrier component and active component are selected from those described in Formulations 1 and 1.1-1.20 herein.

### EXAMPLES

### Example 1—In Vivo Field Study

An in-vivo field study was performed using upside-down jellyfish, family Cassiopeidae. Rubidium iodide and dl-menthol were compared to lanthanide salts and another alkali salt, cesium iodide, for their ability to reduce the dermal irritation following contact with stinging Cnidaria. The base formulation was a commercial SPF15 “TeekaTan™” chemical oil-in-water sunscreen. The null was bare skin. The test subjects reported their perceived stinging sensation from the venomous cassiosomes as no sting (score 0), mild sting (score 1), definite sting (score 2).

| | | | | | DL- | | |
|---|---|---|---|---|---|---|---|
| Skin Location | Control | Null | PrCl₃ | RuI | Menthol | CsI | NdCl₃ |
| Lower arm, left | 0 | | 2 | 0 | 0 | | 2 |
| Upper arm, left | 2 | 2 | 0 | 0 | | 2 | |
| Left calf | | | | 0 | 0 | | |
| Right calf | 0 | 0 | | | | | |
| Stomach | | | | | 0 | | |
| Left side of chest, | | | | | 0 | | |
| underarm | | | | | | | |
| Right side of chest, | | | | | 0 | | |
| underarm | | | | | | | |
| Left cheek (face) | | 2 | | | 0 | | |
| Right cheek (face) | | 2 | | | 0 | | |
| Left arm joint | 2 | 2 | | 0 | | | |
| Right arm joint | | | | | 0.75 | | |
| Left side of lips, upper | | | | | 0 | | |
| and lower | | | | | | | |
| Right side of lips, upper | | 2 | | | | | |
| and lower | | | | | | | |
| Average Sting Score | 1.00 | 1.71 | 1.00 | 0.00 | 0.23 | 2.00 | 2.00 |

This study demonstrated the high selectivity of rubidium, as cesium iodide and lanthanide chlorides did not appreciably reduce the stinging sensation. DL-menthol was the second most effective compound tested.

### Example 2—Second In Vivo Study

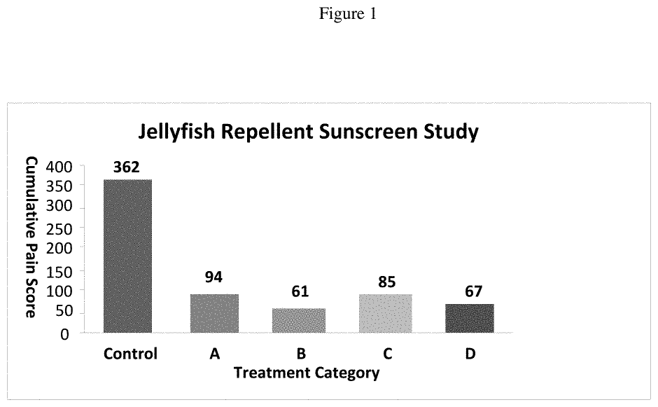

In a second confirmation in-vivo study using upside-down jellyfish in June 2022 with Cassiopea spp. (C. frondosa and C. xamachana), a cumulative pain score was quantified with and without protective compounds in an oil-in-water sunscreen formulation. Bare skin was evaluated against formulations with varying amounts of protective compounds. Forty-eight volunteers were tested after waivers, a review, and compensation procedures. A controlled volume of 1 mL of each formulations was applied in a randomized manner to positions on one or both forearms with palms facing up. The formulation was rubbed into the skin by the clinician wearing rubber gloves and then allowed to dry for 5 minutes. A single excised jellyfish tentacle was then applied to each test site using forceps, and a pain perception score was assigned within 5 minutes. The results are shown in FIG. 1. This study found that the inclusion of 1% w/w rubidium iodide, dl-menthol, and a combinations of these two compounds (Samples A, B, C, respectively) provided significantly reduced pain scores as compared to unprotected skin (control).

Rubidium is a biological analog to potassium. While not wishing to be bound by any particular theory, it is believed that rubidium iodide's effectiveness is correlated to the rubidium ion's ability to permeate the nematocyst cytoplasm wall and displace potassium ions there, interfering with the discharge mechanism which is largely mediated by calcium ions. Monoterpenoids are known calcium (Ca⁺²) ion blockers and may deactivate the calcium-mediated triggering of the nematocyst. See R. Lubbock, B. L. Gupta, T. A. Hall. Novel role of calcium in exocytosis: mechanism of nematocyst discharge as shown by x-ray microanalysis. Proceedings of the National Academy of Sciences June 1981, 78 (6) 3624-3628; DOI: 10.1073/pnas.78.6.3624, incorporated herein by reference for all purposes. In addition, C₇-C₉ aliphatic alcohols appear to have an anesthetizing effect on Cnidarians in captivity this may be a contributing factor to reduced nematocyst discharge.

### Example 3—Skin Moisturizer Formulations

Skin moisturizer formulations in accordance with the present invention are prepared having the following composition:

| | Formulation | Formulation | Formulation |
|---|---|---|---|
| | 1 | 2 | 3 |
| | Amount | Amount | Amount |
| Ingredient | (% w/w) | (% w/w) | (% w/w) |
| RuI | 0.01-3.0 | | |
| DL Menthol | | 0.01-5.0 | |
| n-heptanol | | | 0.01-3.0 |
| Water | 50-99 | 50-99 | 50-99 |
| Humectant; e.g. Glycerin, etc. | 0.01-40 | 0.01-40 | 0.01-40 |
| Emollient or moisturizer: e.g. | 1.0-15.0 | 1.0-15.0 | 1.0-15.0 |
| one or more of carprylic/capric | | | |
| triglyceride, dimethicone, | | | |
| hyaluronic acid, hydrolyzed | | | |
| hyaluronic acid, polyglyceryl | | | |
| diisostearate, ceteareth-20, | | | |
| behentrimonium methosulfate. | | | |
| Surfactant, emulsifier and/or | 0.02-10.0 | 0.02-10.0 | 0.02-10.0 |
| stabilizer: e.g. one or more of | | | |
| cetearyl alcohol, cetyl alcohol, | | | |
| carbomer, sodium lauroyl | | | |
| lactylate, xanthan gum, | | | |
| polysorbate 20. | | | |
| pH adjuster/buffer: potassium | 0.01-10 | 0.01-10 | 0.01-10 |
| phosphate/dipotassium phosphate, | | | |
| buffers based on carbonate, | | | |
| citrate, etc. | | | |
| Skin replenisher: e.g. one or more | 0.01-10 | 0.01-10 | 0.01-10 |
| of ceramide NP, ceramide AP, | | | |
| ceramide EOP, phytosphingosine. | | | |
| Preservatives: e.g. parabens such | 0.01-10 | 0.01-10 | 0.01-10 |
| as methylparaben and/or | | | |
| propylparaben, phenoxyethanol, | | | |
| ethylhexylglycerin | | | |
| optionally, a chelating agent: e.g. | 0.01-10 | 0.01-10 | 0.01-10 |
| EDTA. | | | |

The formulations are expected to significantly reduce the dermal irritation following contact with stinging Cnidaria, in addition to providing efficacy as moisturizers.

### Example 4—Sunscreen Formulations

Sunscreen formulations in accordance with the present invention are prepared having the following composition:

| | Formulation 4 | Formulation 5 | Formulation 6 |
|---|---|---|---|
| | Amount | Amount | Amount |
| Ingredient | (% w/w) | (% w/w) | (% w/w) |
| RuI | 0.01-3.0 | | |
| DL Menthol | | 0.01-5.0 | |
| n-heptanol | | | 0.01-3.0 |
| Water | 30-99 | 30-99 | 30-99 |
| UV absorber: e.g., | 20-40 | 20-40 | 20-40 |
| one or more of | | | |
| PABA, avobenzone, | | | |
| homosalate, | | | |
| octisalate, | | | |
| octocrylene | | | |
| Humectant; e.g. | 0.01-40 | 0.01-40 | 0.01-40 |
| Glycerin, etc. | | | |
| Thickening/ | 0.01-40 | 0.01-40 | 0.01-40 |
| absorbent/anti-caking | | | |
| agent: e.g. aluminum | | | |
| starch octenylsuccinate, | | | |
| arachidyl alcohol, | | | |
| acrylates/C10-30 | | | |
| alkyl acrylate | | | |
| crosspolymer | | | |
| Film forming | 0.01-20 | 0.01-20 | 0.01-20 |
| polymer: e.g. styrene/ | | | |
| acrylates copolymer, | | | |
| beeswax, etc. | | | |
| Emollient or | 0.01-10.0 | 0.01-10.0 | 0.01-10.0 |
| moisturizer: e.g. one | | | |
| or more of carprylic/ | | | |
| capric triglyceride, | | | |
| dimethicone, | | | |
| hyaluronic acid, | | | |
| hydrolyzed | | | |
| hyaluronic acid, | | | |
| polyglyceryl | | | |
| diisostearate, | | | |
| ceteareth-20, | | | |
| behentrimonium | | | |
| methosulfate, | | | |
| isododecane, | | | |
| neopentyl glycol | | | |
| diheptanoate, behenyl | | | |
| alcohol | | | |
| Surfactant, Emulsifier | 0.02-10.0 | 0.02-10.0 | 0.02-10.0 |
| and/or stabilizer: e.g. | | | |
| one or more of | | | |
| cetearyl alcohol, cetyl | | | |
| alcohol, carbomer, | | | |
| sodium lauroyl | | | |
| lactylate, xanthan | | | |
| gum, polysorbate 20 | | | |
| pH adjuster/buffer: | 0.01-10 | 0.01-10 | 0.01-10 |
| potassium phosphate/ | | | |
| dipotassium | | | |
| phosphate, buffers | | | |
| based on carbonate, | | | |
| citrate, etc. | | | |
| Skin replenisher: e.g. | 0.01-10 | 0.01-10 | 0.01-10 |
| one or more of | | | |
| ceramide NP, | | | |
| ceramide AP, | | | |
| ceramide EOP, | | | |
| phytosphingosine. | | | |
| Preservatives: e.g. | 0.01-10 | 0.01-10 | 0.01-10 |
| parabens such as | | | |
| methylparaben and/or | | | |
| propylparaben, | | | |
| phenoxyethanol, | | | |
| ethylhexylglycerin | | | |
| optionally, a chelating | 0.01-10 | 0.01-10 | 0.01-10 |
| agent; e.g. EDTA. | | | |

The formulations are expected to significantly reduce the dermal irritation following contact with stinging Cnidaria, in addition to providing efficacy as sunscreens.

While the invention has been described in connection with what is presently considered to be the most practical and preferred embodiment, it is to be understood that the invention is not to be limited to the disclosed embodiments, but to the contrary, it is intended to cover various modifications or equivalent arrangements included within the spirit and scope of the appended claims. The scope is to be accorded the broadest interpretation so as to encompass all such modifications and equivalent structures as is permitted under the law.

Each of the patents, books, articles and other printed publications referenced herein are incorporated by reference in their entireties for all purposes.

1. A topical formulation for protecting a mammal against Cnidaria toxins comprising:
at least about 0.01% w/w of an active component, the active component comprising one or more compounds independently selected from monocyclic monoterpenoids; rubidium salts;
and C₇-C₉ aliphatic alcohols; and
a carrier component for maintaining the active component on the skin of the mammal.

2. The topical formulation of claim 1, wherein the active component comprises one or more compounds selected from:
a monocyclic monoterpenoid selected from DL menthol, α-terpineol, limonene, thymol, menthol, carvone, eucalyptol, perillaldehyde, p-menthane, an α, β, γ, δ-terpinene, and hinokitiol in a concentration of from about 0.01% w/w to about 5.0% w/w, e.g. from about 0.10% w/w to about 5.0% w/w, e.g. from about 0.01% w/w to about 3.0% w/w, e.g. from about 0.25% w/w to about 1.5% w/w, e.g. about 0.25% w/w, about 0.50% w/w, about 0.75% w/w, about 1.0% w/w, about 1.25% w/w or about 1.50% w/w;
a rubidium halide salt in a concentration of from about 0.01% w/w to about 3.0% w/w, e.g. from about 0.10% w/w to about 3.0% w/w, e.g. from about 0.50% w/w to about 1.5% w/w, e.g. about 0.50% w/w, about 0.75% w/w, about 1.0% w/w, 1.25% w/w or about 1.50% w/w; and
a C₇-C₉ aliphatic alcohol selected from n-heptanol, n-octanol and n-nonanol in a concentration of from about 0.01% w/w to about 3.0% w/w, e.g. from about 0.10% w/w to about 3.0% w/w, e.g. from about 0.25% w/w to about 1.5% w/w, e.g. about 0.25% w/w, about 0.50% w/w, about 0.75% w/w, about 1.0% w/w, about 1.25% w/w or about 1.50% w/w.

3. The topical formulation of claim 2, wherein the active component comprises one or more compounds selected from:
DL menthol;
rubidium iodide; and
a C₇-C₉ aliphatic alcohol selected from n-heptanol, n-octanol and n-nonanol.

4. The topical formulation of claim 1, wherein the active component comprises rubidium iodide.

5. The topical formulation of claim 1, wherein the active component comprises DL menthol.

6. The topical formulation of claim 1, wherein the active component comprises one or more of n-heptanol, n-octanol and n-nonanol.

7. The topical formulation of claim 1, wherein the active component comprises from about 0.01% w/w to about 90% w/w of the formulation.

8. The topical formulation of claim 1, wherein the active component comprises a combination of a monocyclic monoterpenoid selected from DL menthol, α-terpineol, limonene, thymol, menthol, carvone, eucalyptol, perillaldehyde, p-menthane, an α, β, γ, δ-terpinene, and hinokitiol; and a rubidium halide salt selected from rubidium iodide, rubidium chloride, rubidium bromide, and rubidium fluoride.

9. The topical formulation of claim 1, wherein the active component comprises a combination of a monocyclic monoterpenoid selected from DL menthol, α-terpineol, limonene, thymol, menthol, carvone, eucalyptol, perillaldehyde, p-menthane, an α, β, γ, δ-terpinene, and hinokitiol; and a C₇-C₉ aliphatic alcohol elected from n-heptanol, n-octanol and n-nonanol.

10. The topical formulation of claim 1, wherein the active component comprises a combination of a rubidium halide salt and a C₇-C₉ aliphatic alcohol n-heptanol, n-octanol and n-nonanol.

11. The topical formulation of claim 8, wherein the monocyclic monoterpenoid comprises DL menthol, and the rubidium halide salt comprises rubidium iodide.

12. The topical formulation of claim 9, wherein the monocyclic monoterpenoid comprises DL menthol, and the C₇-C₉ aliphatic alcohol comprises one or more of n-heptanol, n-octanol and n-nonanol.

13. The topical formulation of claim 10, wherein the rubidium halide salt comprises rubidium iodide, and the C₇-C₉ aliphatic alcohol comprises one or more of n-heptanol, n-octanol and n-nonanol.

14. (canceled)

15. The topical formulation of claim 14, wherein the carrier component comprises an oil-in-water emulsion, or a nonaqueous (oil) phase.

16. (canceled)

17. The topical formulation of claim 1, wherein the carrier component is selected from a skin moisturizer formulation; an oil-in-water emulsion optionally comprising one or more sunscreen compounds; a dimethicone-based gelee (wax) formulation; an after-sun lotion, a “chemical free” or mineral sunscreen, a sprayable anhydrous (oil and alcohol) formulation, a sprayable milk, and a balm.

18. The topical formulation of claim 1, wherein:
the formulation comprises an aqueous (water) phase and a non-aqueous phase;
the carrier component is a skin moisturizer formulation; and
the active component comprises rubidium iodide at 0.5% w/w to 1.0% w/w in the water phase.

19. The topical formulation of claim 1, wherein:
the carrier component is an oil-in-water emulsion;
the active component comprises rubidium iodide at 0.5% w/w to 1.5% w/w; e.g. 1.0% w/w in the water phase; and
the carrier component optionally contains one or more sunscreen compounds.

20. The topical formulation of claim 1, wherein:
the carrier component is a dimethicone-based gelee (wax) formulation;
the active component comprises DL menthol and a C₇-C₉ aliphatic alcohol selected from one or more of n-heptanol, n-octanol and n-nonanol incorporated into the carrier component.

21. The topical formulation of claim 1, wherein:
the carrier component is an after-sun lotion comprising an aqueous (water) phase and a non-aqueous (i.e., oil) phase;
the active component comprises rubidium iodide at 0.5% w/w to 1.5% w/w; e.g. 1.0% w/w in the water phase, and DL menthol at 0.2% w/w to 1.0% w/w; e.g. 0.5% w/w in the oil phase.

22. A method for protecting a mammal against Cnidaria toxins comprising applying a composition according to claim 1 to the skin of the mammal.

23. (canceled)

24. The method of claim 22 wherein the mammal is a human.

25. (canceled)

26. A method for preparing a topical formulation for protecting a mammal against Cnidaria toxins comprising: providing a carrier component; and
combing the carrier component with at least about 0.01% w/w of an active component, the active component comprising one or more compounds independently selected from monocyclic monoterpenoids; rubidium salts; and C₇-C₉ aliphatic alcohols.

27. The method of claim 26, wherein:
the active component comprises one or more compounds selected from:
a monocyclic monoterpenoid selected from DL menthol, α-terpineol, limonene, thymol, menthol, carvone, eucalyptol, perillaldehyde, p-menthane, an α, β, γ, δ-terpinene, and hinokitiol in a concentration of from about 0.01% w/w to about 5.0% w/w, e.g. from about 0.10% w/w to about 5.0% w/w, e.g. from about 0.01% w/w to about 3.0% w/w, e.g. from about 0.25% w/w to about 1.5% w/w, e.g. about 0.25% w/w, about 0.50% w/w, about 0.75% w/w, about 1.0% w/w, about 1.25% w/w or about 1.50% w/w;
a rubidium halide salt in a concentration of from about 0.01% w/w to about 3.0% w/w, e.g. from about 0.10% w/w to about 3.0% w/w, e.g. from about 0.50% w/w to about 1.5% w/w, e.g. about 0.50% w/w, about 0.75% w/w, about 1.0% w/w, 1.25% w/w or about 1.50% w/w; and
a C₇-C₉ aliphatic alcohol selected from n-heptanol, n-octanol and n-nonanol in a concentration of from about 0.01% w/w to about 3.0% w/w, e.g. from about 0.10% w/w to about 3.0% w/w, e.g. from about 0.25% w/w to about 1.5% w/w, e.g. about 0.25% w/w, about 0.50% w/w, about 0.75% w/w, about 1.0% w/w, about 1.25% w/w or about 1.50% w/w;
and
the carrier component:
comprises an aqueous (water) phase and a nonaqueous (oil) phase; or
comprises an oil-in-water emulsion; or
comprises or consists of a nonaqueous (oil) phase; or
the carrier component is selected from a skin moisturizer formulation; an oil-in-water emulsion optionally comprising one or more sunscreen compounds; a dimethicone-based gelee (wax) formulation; an after-sun lotion, a “chemical free” or mineral sunscreen, a sprayable anhydrous (oil and alcohol) formulation, a sprayable milk, and a balm.
